(19) United States
(12) Patent Application Publication
(10) Pub. No.: US 2004/0105931 A1
(43) Pub. Date: Jun. 3, 2004

(54) Enzymatic modification of sterols using sterol-specific lipase

(76) Inventors: Sobhi Basheer, Sachnin (IL); Dorit Plat, Adi (IL)

Correspondence Address: BANNER & WITCOFF, 1001 G STREET N W, SUITE 1100, WASHINGTON, DC, 20001, US

(21) Appl. No.: 10/240,546

(22) PCT Filed: Apr. 3, 2001
(86) PCT No.: PCT/IL01/00305

(30) Foreign Application Priority Data
Apr. 4, 2000 (IL) 135466

(51) Int. Cl. A23D 7/00

(52) U.S. Cl. 426/601

(57) ABSTRACT

The invention relates to a process for the selective alcoholysis of a free sterol, by contacting said free sterol with a fat-based product, optionally with the addition of carboxylic fatty acid(s) and/or ester derivative(s) thereof that are not derived from said fat-based product, in the presence of an immobilized lipase complex which may optionally be surfactant-coated, which complex possesses a high level of sterol-specific alcoholytic and/or esterfication activity and minimal acidolytic and transesterification activities. The fat-based product is a nutritional product or food, particularly butterfat, or a cosmetic or cosmetic or cosmetic-related product. The process may be used for preparing substantially cholesterol-free fat-based products, particularly products containing butterfat, by selectively esterfying any free cholesterol contained therein by the immobilized, preferably surfactant coated lipase. The invention also relates to a process for the in situ enrichment of a fat-based product with esterified phytosterol ester(s). In this process, the esterification of the phytosterol is simultaneously accompanied by esterification of any free cholesterol present in said fat-based product.

### FIELD OF THE INVENTION

The present invention is concerned with the use of lipases for the selective alcoholysis and/or esterification of sterols. More specifically, the invention relates to the use of lipases for the selective alcoholysis and/or esterification of free cholesterol in foods, particularly in butterfat. The present invention further relates to a process for in situ enriching a fat-based product with esterified phytosterol ester(s) and simultaneously depleting the free cholesterol content of said fat-based product.

### BACKGROUND OF THE INVENTION

Sterols are alcohols containing a steroid nucleus linked to an 8 to 10-carbon side-chain and a hydroxy group. Such compounds have a wide distribution in the plant and animal kingdoms. The most prevalent plant sterol is ergosterol, while the animal sterol of greatest significance is cholesterol.

In recent years it has been increasingly recognized that certain lipid components present in food, including cholesterol, constitute significant risk factors in the pathogenesis of atherosclerotic disease. As a result of research in this field, there have been many attempts to develop methods for modifying the composition of many widely consumed food products, such that the levels of potentially harmful lipid and lipid-like substances are reduced. Of particular prominence in this field are those studies directed at reducing the cholesterol content of food products. In this regard, there is a general interest in developing methods of modifying important dietary fats, such as butterfat, for use in the preparation of low- or zero-cholesterol containing foodstuffs such as butter and ice cream.

Japanese published Patent Application No. 42944/71 discloses a method for obtaining cholesterol-reduced substances by means of extraction of cholesterol therefrom with organic solvents such as acetone or hexane.

Another method of reducing cholesterol levels in food products is the adsorption of cholesterol therefrom, for example by the use of polymer-supported digitonin [J. Agric. Food Chem. 38:1839 (1990)].

An alternative to the removal of cholesterol from food is to convert free cholesterol to a less harmful or harmless derivative. U.S. Pat. No. 4,921,710, for example, discloses the use of cholesterol reductase in the conversion of cholesterol to coprostanol.

Another modification process that has been described is the enzymatic conversion of free cholesterol to form a cholesterol-fatty acid ester. In one report [Kalo, P. et al., 1993, Fat Sci. Technol. 95:58-62], the formation of cholesterol esters during lipase-catalyzed interesterification reactions was noted. The cholesterol modification reported in that study was a by-product of the main reaction directed towards obtaining desired modifications in the triglyceride composition.

It is an object of the invention to provide a method for the modification of sterols, particularly harmful dietary sterols such as cholesterol.

It is a further object of this invention to provide a method for the neutralization of cholesterol in butterfat.

It is another object of the invention to provide a method for the selective neutralization of cholesterol without affecting other lipid components (triglycerides) present in the butterfat.

It is yet another object of the invention to provide a process for in situ enriching a fat-based product, preferably nutritional and cosmetic-related products, with esterified phytosterol ester(s), while at the same time depleting the cholesterol content of said fat-based product.

These and other objects and advantages of the invention will become apparent as the description proceeds.

### SUMMARY OF THE INVENTION

It has now been surprisingly found that certain immobilized, optionally surfactant-coated lipase preparations may be used for the selective alcoholysis and/or esterification of sterols. While the selective alcoholysis and/or esterification process described below is applicable to many different sterols, be they of animal, plant or synthetic origin, the major application of the process of the invention is the selective alcoholysis and/or esterification of dietary cholesterol and phytosterols.

The invention is primarily directed to a process for the selective alcoholysis and/or esterification of free sterols, comprising contacting said sterols with a complex of an immobilized lipase, which may be optionally surfactant-coated, the complex possessing a high level of alcoholytic and/or esterification activity coupled with minimal acidolytic and interesterification activities, for a time sufficient for preferably maximal alcoholysis and/or esterification of said free sterols to occur, following which the optionally coated immobilized lipase complex and the esterified cholesterol are removed, if so desired.

The process of the invention comprises alcoholysis and/or esterification of sterols using immobilized lipase, optionally surfactant-coated, in natural oils and fats media—as follows:

1. Esterification:

Sterol-OH+ added fatty acid (or ester derivative thereof) to a natural oil and fat medium, to give esterified sterol.

2. Alcoholysis:

Sterol-OH+ triglycerides present in a natural oil and fat medium (for example, olive oil) to give sterol-fatty acyl ester+diacyl-glycerol.

The invention is particularly directed to a process for the selective alcoholysis and/or esterification of free cholesterol, comprising contacting the free cholesterol with a surfactant-coated, insoluble matrix immobilized lipase complex possessing a high level of alcoholytic and/or esterifying activity coupled with minimal acidolytic and interesterification activities, for a time sufficient for maximal alcoholysis of said free cholesterol to occur, following which the esterified cholesterol is removed from said lipase complex.

By the term “selective alcoholysis” is meant that the above-described process causes alcoholysis or esterification of the free cholesterol, without causing a significant change in the identity or positional distribution of the fatty acyl groups on the glycerol backbone of the triglycerides present in the fat-based product, particularly butterfat.

In a preferred embodiment of the invention, the free cholesterol to be esterified is present in butterfat.

In a preferred embodiment of the invention, the alcoholysis and/or esterification reaction is carried out in a microaqueous environment. Preferably, the microaqueous environment is provided by an organic solvent. While any suitable organic solvent may be used, a preferred solvent is n-hexane. In another preferred embodiment of the invention, the alcoholysis and/or esterification reaction is performed in a solvent-free system.

In another preferred embodiment of the process of the invention, the lipase is selected from the group consisting of Candida antarctica, Candida antarctica A and Candida rugosa. In a still more preferred embodiment, the lipase is either Candida antarctica A or Candida rugosa.

The invention also encompasses a process for preparing substantially cholesterol-free butterfat-containing products, comprising alcoholysis and/or esterifying the cholesterol contained in a butterfat product precursor with a lipase. In one embodiment of the invention, the lipase used in this process is immobilized onto an insoluble matrix. In another preferred embodiment of the invention the lipase is surfactant-coated. In a further, more preferred embodiment the lipase is immobilized onto an insoluble matrix and also surfactant coated. In a yet further embodiment of the invention, the modified (surfactant-coated)-immobilized lipase complex is used in a granular form resulted in a considerable enhancement of stability as well as permitting the repeat of enzyme use, without loss of activity.

In another aspect, the invention is also directed to a modified, low cholesterol butterfat composition wherever produced by the process of the invention.

The present invention is also directed to low-cholesterol food preparations containing modified butterfat compositions, whenever the latter are produced by the process of the invention. In a preferred embodiment of this aspect of the invention, the low-cholesterol food preparations containing modified butterfat compositions are selected from the group consisting of low-cholesterol butter, cocoa butter, ice cream, coffee whiteners and creams, cheeses, other dairy products and other sterol-containing foods.

In a yet additional preferred embodiment of the invention, the above low-cholesterol food preparations are further enriched with esterified plant sterols, such as stigmasterol, and others.

In a further preferred embodiment, the invention provides a process for the enrichment of cosmetics and/or cosmetics-related products with esterified plant sterols, such as stigmasterol, and others.

While the processes of the invention may be performed using any suitable lipase or lipase preparation, in a preferred embodiment, the source of said lipase or lipase preparation is selected from the group consisting of Aspergillus niger, Candida antarctica, Candida cylindracea, Mucor miehei, Pseudomonas cepacia, Rhizopus niveus, Rhizopus arrhizus, Hog pancreas, Aspergillus oryzae, Candida lipolytica, Mucor javanicus, Penicillium roqueforti, Pseudomonas fluorescens, Rhizomucor miehei, Wheat germ, Chromobact. viscosum, Lipoprotein from Pseudomonas species, Lipoprotein from Pseudomonas species B, Lilipase A-10FG, Saiken 100, Lipase G, Lipase M, Lipase F-AP-15, Newlase F, Pancreatin F, Newlase AP6, Lipase AY, Lipase PS, Novozym 525, Novozym 868, Novozym 388, Novozym 398, Novozym 435 (immobilized), Lipozyme IM-60 (immobilized) and PPL.

All the above and other characteristics and advantages of the invention will be further understood from the following illustrative and non-limitative figures and examples of preferred embodiments thereof.

### BRIEF DESCRIPTION OF THE DRAWINGS

The present invention will be more clearly understood from the detailed description of the preferred embodiments and from the attached drawings in which:

FIG. 1 is a typical gas chromatogram for untreated anhydrous milk fat (AMF).

FIG. 2 is a typical gas chromatogram for AMF following treatment with Enzymoalc in microaqueous n-hexane.

FIG. 3 is a typical gas chromatogram for AMF-triglyceride fraction before and after the second treatment with the enzyme.

FIG. 4 is a typical gas chromatogram for AMF enriched with stigmasterol (peak at 5.10) and with cholesterol (peak at 4.75) as an internal standard.

FIG. 5 is a typical gas chromatogram for a sample of AMF enriched with stigmasterol following treatment with Enzymoalc 7C.

FIG. 6 is a typical gas chromatogram for a sample of olive oil enriched with phytosterols following treatment with 4A.

FIG. 7 represents a conversion of stigmasterol to stigmasterol oleate in olive oil in the presence of hexane using enzymes preparations 4B, 4C, 4E and 4F.

FIG. 8 is a typical gas chromatogram for a sample of AMF enriched with stigmasterol (peak at 4.84). The cholesterol peak appears at 4.39.

FIG. 9 is a typical gas chromatogram for the stigmasterol enriched AMF following treatment with Enzymoalc 4C.

### DETAILED DESCRIPTION OF PREFERRED EMBODIMENTS

For purposes of clarity and as an aid in the understanding of the invention, as disclosed and claimed herein, the following terms and abbreviations are defined below:

Cholesterol neutralization: the conversion of free cholesterol to a cholesterol ester.

Low cholesterol: this term is used to indicate that the food or composition contains a low level of free cholesterol

Enzymoalc: represents a modified, immobilized enzyme, unless otherwise indicated.

A-G: different types of enzymatic preparations described at the following Table 1.

TABLE 1

| Enzyme | | |
|---|---|---|
| Preparation | Modification | Immobilization |
| A | SMS | Celite |
| B | SMS | Silica Sipernat 700 |
| C | SMS | Silica Sipernat D17 |
| D | SMS | Silica Aerosil R972 |
| E | SMS | Silica Sipernat 22 |
| F | SMS | Silica Sipernat 630 |
| G | SMO | Duolite |

SMS - sorbitol mono-stearate;
SMO - sorbitol mono oleate.

General Methods

1. Modification of the Enzyme

The modified (surfactant-coated) lipase is prepared as described in WO99/15689, which is herein fully incorporated by reference.

Briefly, the crude enzyme (300 mg/l protein) is dissolved in 1 L Tris buffer, pH 5.5 containing 4 g insoluble inorganic or organic matrix (Celite, silica gel, alumina or polypropylene). The solution is stirred vigorously with a magnetic stirrer for 30 minutes at 10° C. In the case of surfactant-coated immobilized enzyme preparations, sorbitan mono-stearate (1 g dissolved in 20 ml ethanol) is added drop-wise to the stirred enzyme solution. All enzyme preparations (i.e. both the surfactant-coated immobilized lipases and the immobilized-crude lipases) are sonicated for 10 minutes and then stirred for 3 hours at 10° C. The formed precipitate is collected by either filtration or centrifugation (12,000×rpm, 4° C.), followed by overnight freezing at −20° C. and lyophilization.

The granulation process of the modified-immobilized lipase complex is performed using various binding reagents such as starch, methyl- or ethyl-cellulose, gums, agarose or other binders, as described in WO 99/15689.

2. Enzymatic Reactions

2.1 Standard Esterification Reaction Conditions

Unless stated otherwise, all reactions in the Examples given below were carried out under the following conditions:

Lipase (1 mg protein), in a crude, immobilized or modified-immobilized form, was added to a solution (1) of n-hexane containing cholesterol (20 mg) and stearic acid (20 mg). The reaction mixture was stirred for 16 hours at 40° C. Samples were taken from the reaction mixture, filtered though 0.45 μm membrane filters and analyzed by gas chromatography (GC).

It should be noted that the addition of lipase preparation to the cholesterol in n-hexane, or solvent-free form, in the presence of stearic acid (or any other fatty acid), is for the selection of a lipase enzyme possessing the optimal esterification activity. For performing the reaction in a natural oil and fat medium, there is no need for addition of external cholesterol to the reaction.

2.2 Alcoholysis Reactions in a Medium of Anhydrous Milk Fat (AMF)

2.2.1 Neutralization of the Enriched Cholesterol in AMF

(A) 10 mg lipase (in a crude, immobilized or modified-immobilized form) were added to 1 ml of n-hexane containing AMF (400 mg) and free cholesterol (20 mg). The reaction mixture was shaken at 60° C. for 14 hours. At pre-determined time intervals, samples were taken from the reaction mixtures, diluted appropriately with n-hexane and injected into a gas chromatography system.

(B) 50 mg lipase (in a crude, immobilized or modified-immobilized form) was added to 1 g AMF. The reaction mixture was shaken at 60° C. for 14 hours. At pre-determined time intervals, samples were taken from the reaction mixtures, diluted appropriately with n-hexane and injected into a gas chromatography system.

It should be noted that the addition of cholesterol in n-hexane or solvent-free form to AMF is for illustration only. For performing the reaction in a natural oil and fat medium, there is no need for addition of external cholesterol to the reaction.

2.2.2 Alcoholysis of Stigmasterol in Pure AMF

100 mg of a lipase preparation were added to 1.2 g AMF enriched with 50 mg stigmasterol. The reaction mixture was then heated for 14 hours at 60° C. Samples were taken at pre-determined time intervals, diluted with n-hexane (20 mg reaction mixtures/1 ml n-hexane) and injected into a gas chromatography system.

2.3 Alcoholysis Reactions in Olive Oil

2.3.1 Alcoholysis of Stigmasterol in Olive Oil in an Organic Solvent or in Organic Solvent-Free System

(A) 100 of a lipase preparation were added to 0.5 ml olive oil enriched with 50 mg stigmasterol in 1.5 ml n-hexane. The reaction mixture was then heated for 14 hours at 60° C. Samples were taken at pre-determined time intervals, diluted with n-hexane (50 μl reaction mixture/300 μl n-hexane) and injected into a gas chromatography system.

(B) 100 mg of a lipase preparation were added to 1 ml olive oil enriched with 30 stigmasterol. The reaction mixture was then heated for 14 hours at 60° C. Samples were taken at predetermined time intervals, diluted with n-hexane (40 ml reaction mixture/600 ml n-hexane) and injected into a gas chromatography system.

3. Gas Chromatography

The concentrations of cholesterol and its fatty acid esters, fatty acids, and triglycerides were determined by gas chromatography, using HP-5890, equipped with a flame ionization detector. A capillary column, Ultra-1 (HP) was used under the following separation conditions:

For the AMW samples: The injector and detector temperatures were maintained at 365° C., initial column temperature 150° C., followed by a 1 min isotherm; thereafter, the oven temperature was raised at a rate of 20° C./min to 365° C.

For the olive oil samples: The injector and detector temperatures were maintained at 365° C., initial column temperature 200° C., followed by a 1 min isotherm; thereafter, the oven temperature was raised at a rate of 25° C./min to 365° C.

4. TLC Identification Spray Solution

The dyeing solution was prepared by mixing 5 ml acetic acid, 16.8 ml sulfuric add and 453 ml ethanol 11.3 ml of para-anisaldehyde was added to the solution and the reagent was kept at a sealed container.

5. Chromatographic Separation of AMF Sample Treated by Enzymolac 7C

The quantitative assay for determination of free cholesterol was conducted by isolating the cholesterol and the cholesterol esters from the fats on silica-gel column. The weighed fat sample (100 mg) was dissolved in an eluant (see below) and was introduced to a silica gel 60 (activity II-III) column. The column was eluted with 15 column volumes of 2% diethyl ether in hexane; 10 column volumes of 5% diethyl ether in hexane; 10 column volumes of diethyl ether.

The cholesterol was determined in combined fractions III and IV and cholesterol ester was determined in fraction I by gas chromatography.

6. Enzyme Sources

In some of the examples that follow, the lipases used are identified by use of a short-name of the form: Enzymoalcn, the microbial source, commercial name and manufacturer information for these enzymes are given in Table 2.

TABLE 2

| Enzyme | Microbial Source | Commercial Name | Manufacturer |
|---|---|---|---|
| Enzymoalc1 | Pseudomonas spp. | | Fluka |
| | Lipoprotein | | |
| Enzymoalc2 | Pseudomonas | | Fluka |
| | spp. B Lipoprotein | | |
| Enzymoalc3 | Candida antarctica | | Fluka |
| Enzymoalc4 | Candida antarctica | Novozym 868 | Novo Nordisk |
| | A | | |
| Euzymoalc5 | Chromobacterium | Lipase LP | Asahi |
| | viscosum | | |
| Enzymoalc6 | Pseudornonas | Lipase PS | Amano |
| | cepacia | | |
| Enzymoalc7 | Candida rugosa | Lipase AY | Amano |

### EXAMPLE 1

Esterification of Free Cholesterol with Free Fatty Acids

Various crude and modified lipases obtained from different organisms were tested for their ability to esterify pure cholesterol, using the reaction conditions described hereinabove in “General Methods”. The modified lipases were prepared by coating the crude enzyme with sorbitan mono-stearate or other fatty acid sugar esters, essentially as described in the WO99/15689, the contents of which are fully incorporated herein by reference.

In the case of the modified lipases, the esterification reaction was carried out in microaqueous n-hexane or in a solvent-free system. When crude lipases were tested, however, a small amount of water (up to about 1% (w/w) of total solvent) was added. This addition of water to the solvent system generally leads to the initiation of the hydrolysis of triacylglycerol molecules (i.e. fats and oils). Table 3 presents comparative data for the esterification activity of lipases obtained from different organisms when in their crude, and modified states. In this table, the esterification activity of the enzymes is expressed as percent (w/w) conversion of cholesterol to its fatty acid (stearate) ester.

TABLE 3

| | Cholesterol | |
|---|---|---|
| | conversion (%) | Cholesterol conversion (%) |
| Lipase | With crude lipase | With modified lipase |
| Aspergillus niger | 0 | 3 |
| Candida antarctica | 12 | 67 |
| Candida cylindracea | 2 | 0 |
| Mucor miehei | 0 | 0 |
| Pseudomonas cepacia | 0 | 2 |
| Rhizopus niveus | 0 | 2 |
| Rhizopus arrhizus | 0 | 0 |
| Hog pancreas | 0 | 0 |
| Aspergillus oryzae | 0 | 4 |
| Candida lipolytica | 0 | 0 |
| Mucor javanicus | 0 | 0 |
| Pencillium roqueforti | 0 | 0 |
| Pseudomonas | 1 | 7 |
| fluorescens | | |
| Rhizomucor miehei | 0 | 5 |
| Wheat germ | 0 | 0 |
| Chromobact. viscosum | 1 | 63 |
| Lipoprotein from | 16 | 34 |
| Pseudomonas species | | |
| Lipoprotein from | 23 | 71 |
| Pseudomonas species B | | |
| Lilipase A-10FG | 0 | 0 |
| Saiken 100 | 0 | 0 |
| Novozym 525 | 0 | 0 |
| Novozym 868 | 7 | 32 |
| Novozym 388 | 0 | 0 |
| Novozym 398 | 0 | 0 |
| Novozym 435 | 0 | 0 |
| (immobilized) | | |
| Lipozyme IM-60 | 0 | 0 |
| (immobilized) | | |
| PPL | 0 | 3 |

The results in Table 3 dearly show that both of the lipoprotein lipases as well as Lipase PS, Lipase AY, Chromobact. viscosum lipase and Candida antarctica lipase possess significant esterification activity in their crude form, while the other crude enzymes tested either displayed very low activity or were unable to catalyze the esterification reaction. Although nearly all of the lipases tested showed increased activity following modification (surfactant treatment), the most marked increases were seen with Chromobact. viscosum, Novozym 868 and both lipoprotein lipases.

### EXAMPLE 2

Esterification of Free Cholesterol with Free Fatty Acids: Comparison of Crude Immobilized Enzymes and Modified-Immobilized Enzymes

Seven of the most active enzymes (determined in Example 1, above) were chosen for further study. A comparison was made between the cholesterol conversion catalyzed by the crude enzyme when immobilized to a silica matrix and the conversion catalyzed by the surfactant-modified enzyme when similarly immobilized.

The surfactant modification and immobilization steps were carried out essentially as described in co-owned WO 99/15689, the contents of which are incorporated herein by reference. Briefly, the crude enzymes (300 mg/l protein) were dissolved in 1 L Tris buffer, pH 5.5 containing 4 g insoluble inorganic or organic matrix (Celite, silica gel, alumina or polypropylene).

The solution was vigorously stirred with a magnetic stirrer for 30 minutes at 10° C. In the case of the surfactant-treated immobilized enzyme preparations, sorbitan mono-stearate (1 g dissolved in 20 ml ethanol) was added dropwise to the stirred enzyme solution. All enzyme preparations (i.e. both the surfactant-treated-immobilized and the crude enzyme-immobilized enzymes) were sonicated for 10 minutes and then stirred for 3 hours at 10° C. The precipitate was collected by either filtration or centrifugation (12,000×rpm, 4° C.), followed by overnight freezing at −20° C. and lyophilization.

The esterification reaction conditions were as described in Example 1, with the exception that 10 mg of immobilized enzyme were used (giving a final protein content in the reaction mixture of 0.5% (w/w)).

The results of this comparative study are shown in Table 4.

TABLE 4

Cholesterol conversion (%)

| | crude immobilized | modified immobilized |
|---|---|---|
| Lipase | enzyme | enzyme |
| Enzymoalc1 | 16 | 41 |
| Enzymoalc2 | 26 | 77 |
| Enzymoalc3 | 14 | 24 |
| Enzymoalc4 | 9 | 35 |
| Enzymoalc5 | 11 | 67 |
| Euzymoalc6 | 15 | 69 |
| Enzymoalc7 | 11 | 48 |

These results demonstrate that the selected lipases, when modified by both immobilization and surfactant treatment, are much more efficient catalysts for the cholesterol conversion reaction than the crude or immobilized enzymes without the modification process.

### EXAMPLE 3

Demonstration of the Selectivity of Modified-Immobilized Lipases for the Alcoholysis Reaction Between Cholesterol and Triglycerides

In aqueous media, lipases hydrolyze the ester bond of triacylglycerols to form partial glycerides and free fatty acids. Lipases in non-aqueous media are also able to catalyze transesterification reactions either between two different triglyceride molecules (interesterification) or between a triglyceride molecule and a fatty acid (acidolysis). In this process, acyl groups of fatty acids can be exchanged specifically or non-specifically on the glycerol backbone between the two reacting substrates. In order to select enzyme preparations possessing selectivity for alcoholysis, the activities of the 7 enzymes tested in Example 2 were tested in a further two model reactions:

1. Acidolysis reaction between tripalmitin and lauric acid.

2. Alcoholysis reaction between cholesterol and tripalmitin.

1. The Acidolysis Reactions were Carried Out as Follows:

Tripalmitin (4 mg) and lauric acid (4 mg) were dissolved in 1 ml n-hexane. Modified-immobilized lipase (amount of preparation that contain 0.5% (w/w) protein) was added and the reaction mixture was stirred at 40° C. for 4 hours. The results of these experiments, expressed as percentage tripalmitin conversion (w/w) are shown in Table 5.

TABLE 5

| Modified-immobilized lipase | Tripalmitin conversion (%) |
|---|---|
| Enzymoalc | 165 |
| Enzymoalc2 | 42 |
| Enzymoalc3 | 57 |
| Enzymoalc4 | 5 |
| Enzymoalc5 | 23 |
| Enzymoalc6 | 21 |
| Enzymoalc7 | 8 |

Although all of the lipases tested possess acidolytic activity, the lowest such activity was observed with the lipase derived from Candida antarctica A (Novozyme 868, Novo Nordisk, Denmark), Enzymoalc4, and with the lipase derived from Candida rugosa (Lipase AY, Amano, Japan), Enzymoalc7.

The other lipases tested demonstrated higher levels of acidolytic activity, indicating that they are more likely to cause a change in the positional distribution of fatty acids on the glycerol backbone of the triglycerides present in butterfat samples subjected to this treatment.

2. The Alcoholysis Reactions were Carried Out as Follows:

Cholesterol (5 mg) and tripalmitin (5 mg) were dissolved in n-hexane (1 ml). Modified-immobilized lipase (10 mg; protein content 0.5% (w/w)) was added, and the reaction mixture stirred at 40° C. for 4 hours. The results of this investigation are shown in Table 6.

TABLE 6
Tripalmitin conversion in the alcoholysis reaction with
cholesterol in the presence of different lipase preparations

| Modified-immobilized lipase | Tripalmitin conversion (%) |
|---|---|
| Enzymoalc1 | 69 |
| Enzymoalc2 | 68 |
| Enzymoalc3 | 42 |
| Enzymoalc4 | 61 |
| Enzymoalc5 | 32 |
| Enzymoalc6 | 19 |
| Enzymoalc7 | 47 |

It can be seen from Table 6 that lipases Enzymoalc4 and Enzymoalc7, which possess the lowest levels of acidolytic activity (Table 5) display significant levels of alcoholytic activity.

The activities of the 7 selected lipases in the esterification, alcoholysis and acidolysis reactions are summarized in Table 7.

TABLE 7
Summary of the lipase preparation activity in the
esterification, acidolysis and alcoholysis reactions

| Modified-immobilized | Esterification | Alcoholysis | |
|---|---|---|---|
| lipase | activity¹ | activity² | Acidolysis activity³ |
| Enzymoalc1 | 41 | 69 | 65 |
| Enzymoalc2 | 77 | 68 | 42 |
| Enzymoalc3 | 24 | 42 | 57 |
| Enzymoalc4 | 35 | 61 | 5 |
| Enzymoalc5 | 67 | 32 | 23 |
| Enzymoalc6 | 69 | 19 | 21 |
| Enzymoalc7 | 48 | 47 | 8 |

¹Given as the conversion of cholesterol in the esterification reaction between cholesterol and stearic acid.
²Given as the conversion of tripalmitin in the alcoholysis reaction between cholesterol and tripalmitin.
³Given as the conversion of tripalmitin in the acidolysis reaction between stearic acid and tripalmitin.

From the summary shown in Table 7, it may be seen that two enzymes, Enzymoalc4 and Enzymoalc7, possess very low (or zero) acidolytic activity, low to moderate esterification activity and relatively high alcoholysis activity. These two enzymes are thus considered to be particularly suitable for the selective alcoholysis and/or esterification of sterols and were therefore selected for use in the studies described in the following Examples.

### EXAMPLE 4

Lipase-Catalyzed Neutralization of Cholesterol in Pure AMF

Lipase-catalyzed alcoholysis reactions were performed in AMF as follows:

50 mg lipase (in a crude, immobilized or modified-immobilized form) were added to 1 g AMF. This reaction mixture was shaken at 60° C. for 14 hours. At pre-determined time intervals, samples were taken from the reaction mixtures, diluted appropriately with n-hexane and injected into a gas chromatography system (see “General Methods”, above).

During the reaction, the intensity of the cholesterol peak decreased while it was broadened due to overlapping with the formed diglycerides.

In order to follow the reaction course, anhydrous milk fat (AMF) was enriched by the addition of free cholesterol (Sigma Co., St. Louis, USA) at a ratio of 20 mg cholesterol/1 g AMF.

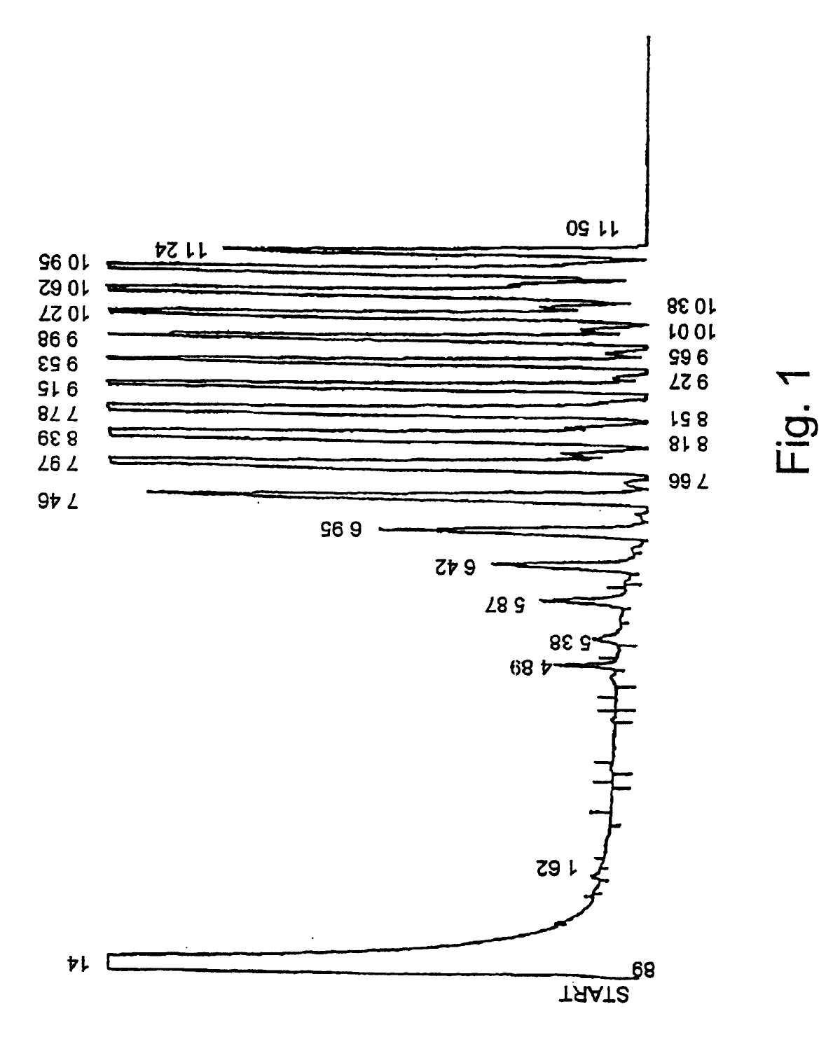

FIG. 1 shows a typical gas chromatogram for pure AMF. The retention time of the cholesterol is 4.89 minutes under the gas chromatography (GC) conditions described at “General methods”. The main peaks in FIG. 1 represent the triglycerides that are present in AMF.

Five different lipase preparations were used in this study—as follows:

10 mg lipase (in a crude, immobilized or modified-immobilized form) were added to 1 ml of n-hexane containing AMF (400 mg) and free cholesterol (20 mg). This reaction mixture was shaken at 40° C. for 14 hours. At pre-determined time intervals, samples were taken from the reaction mixtures, diluted appropriately with n-hexane and injected into a gas chromatography system (see “General Methods”, above). The results of this study are given in Table 8, below, and are expressed as % (w/w) conversion of free cholesterol to cholesterol esters.

TABLE 8
Conversion of cholesterol using six different enzymatic
preparations

| Enzyme | Conversion of |
|---|---|
| preparation | cholesterol (% w/w) |
| Blank (no enzyme was added) | 0 |
| Enzymoalc 4A | 95 |
| Crude Enzymoalc 4 | 0 |
| Crude Enzymoalc 4 immobilized | 60 |
| on silica | |
| Enzymoalc 7A | 98 |
| Crude Enzymoalc 7 | 70 |

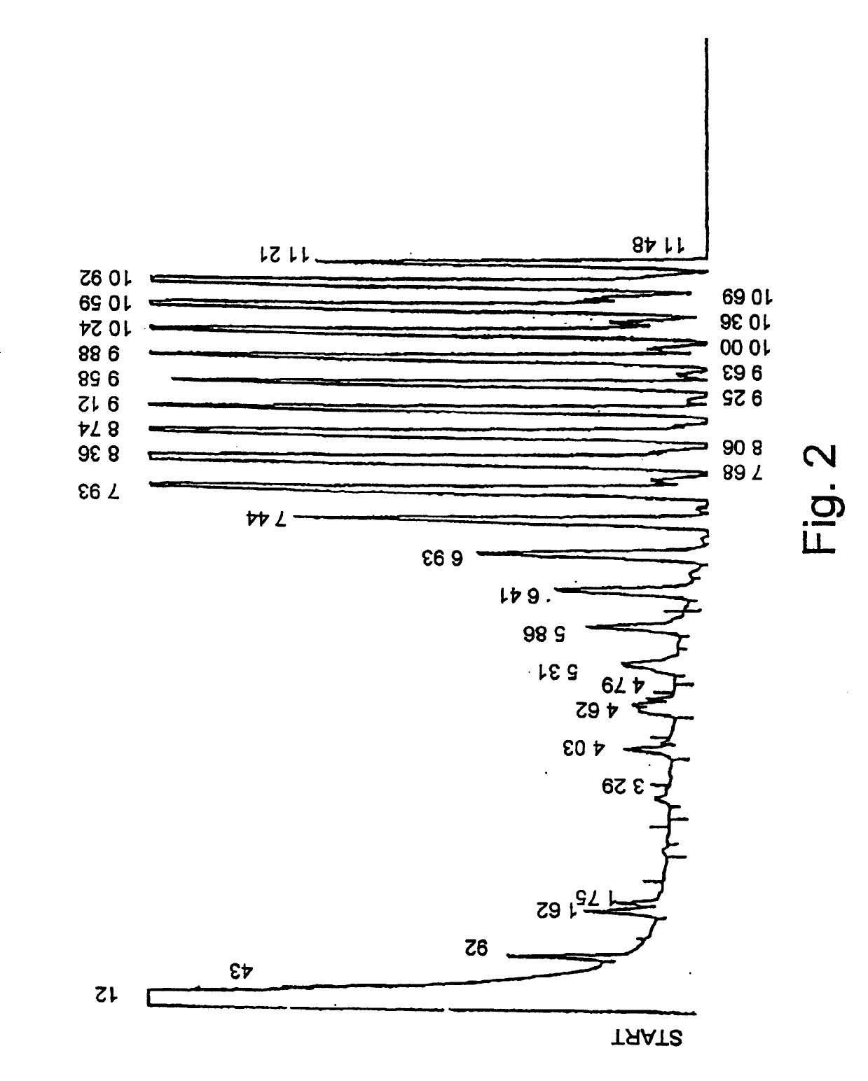

It may be seen from Table 8 that the conversion of cholesterol to its fatty acid esters using the modified-immobilized Enzymoalc4 was around 95%, while 98% of the cholesterol were consumed in the alcoholysis reaction when the AMF was treated with the modified-immobilized Enzymoalc7. The crude enzyme preparation corresponding to the enzyme used in the manufacture of Enzymoalc 4 (Candida antarctica A, see Table 2, above), was inactive in this reaction, while the same crude enzyme when immobilized on silica was able to convert 60% of the free cholesterol into its fatty acid esters. FIG. 2 is a typical gas chromatogram for AMF after treatment with Enzymoalc7 under the reaction conditions described above (with no enrichment with cholesterol). It may be seen from this figure that following reaction, the peak appeared at a retention time of 4.74 minutes decreases significantly and broadens. The reaction course was followed by Thin Layer Chromatography (TLC) performed on Silica Gel plates. A sample of 10 μl was diluted in 500 μl hexane. One microliter from this sample was applied on the plate with a guide spot of cholesterol and cholesterol stearate. The plate was developed in a glass chamber well saturated with solution of 25% ether in hexane. After 7 cm run the developed plate was removed and air-dried. The identification of the spots was conducted using an acidic solution of para-anisaldehyde. During the course of the reaction, spots that fit the cholesterol ester appeared (R_{f} (cholesterol stearate)=0.9) and few new spots appeared at the cholesterol region that belong probably to the di-glycerides formed during the reaction. A careful look at the TLC reveals that there is no spot that fits the cholesterol standard. Furthermore, the color of the cholesterol standard is purple whereas the other spots appeared sandier.

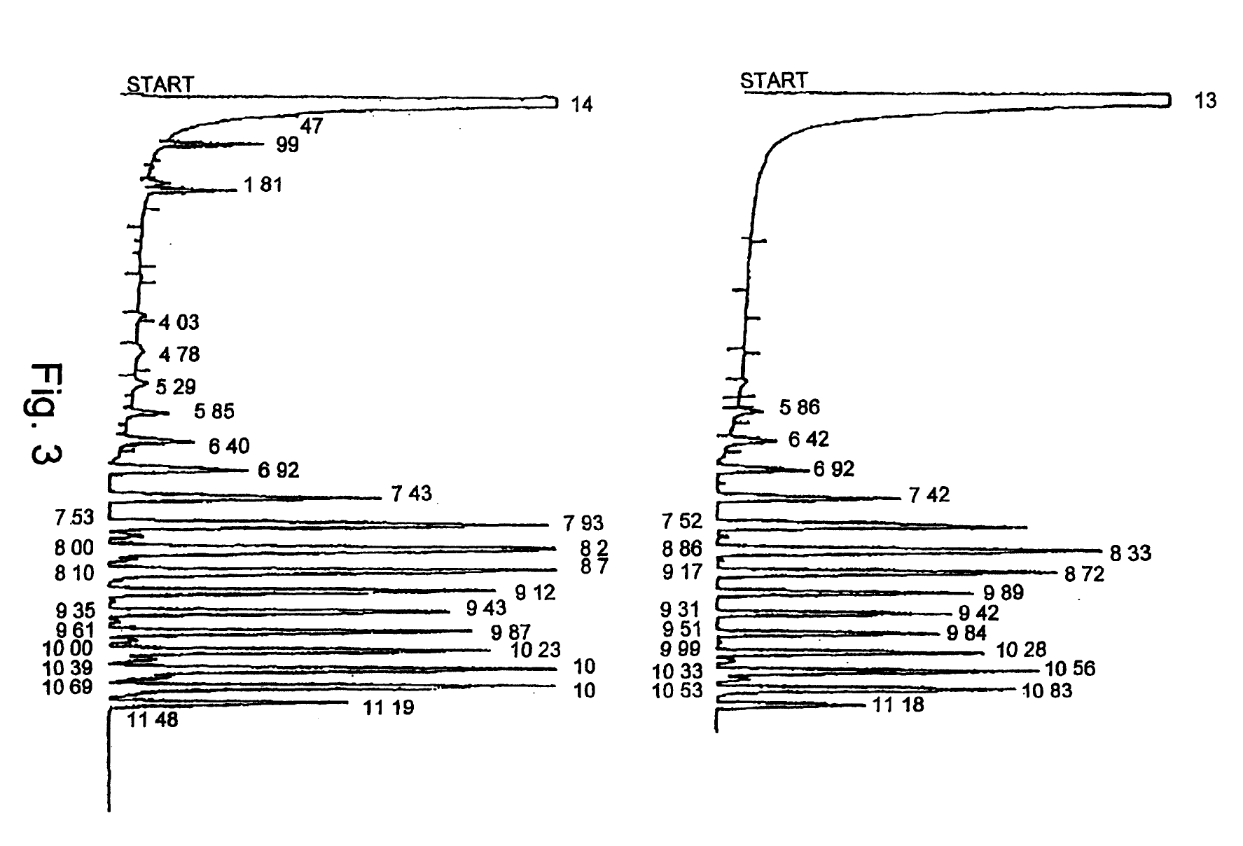

In order to prove that the content of the triglycerides does not change during the reaction course, the product was loaded on column chromatography (silica-gel, hexane-ether). The mixture of esters was eluted with pure hexane, whereas the triglycerides mixture was eluted with a mixture of 2% ether in hexane. The more polar fraction was eluted with pure ether. After combining and evaporating the solvent from the fraction of the triglycerides, they were submitted to a further enzymatic reaction using the same enzyme (enzymoalc 7). After one night reaction, sample of 10 μl was diluted with hexane and injected into the GC. FIG. 3 shows the triglycerides spectra before and after the second enzymatic reaction. It can be seen that there are no significant spectral changes, i.e. the triglycerides content remains with no, or only minor, changes and the enzyme is cholesterol-specific and selective.

### EXAMPLE 5

Enrichment of AMF with Stigmasterol Esters

Lipase-catalyzed alcoholysis of stigmasterol was performed in AMF as follows:

100 mg lipase (in a crude or modified-immobilized form) were added to 1.2 g AMF containing 50 mg stigmasterol. This reaction mixture was shaken at 60° C. for 14 hours. At pre-determined time intervals, samples were taken from the reaction mixture, diluted appropriately with n-hexane and injected into a gas chromatography system (see “General Methods”, above). Cholesterol was added to each of the samples for serving as internal standard. The cholesterol is added to the sample of the reaction mixture after the enzyme was filtered out from the reaction or it was added to the sample taken from the reaction mixture meaning there is no enzyme inside.

The conversion was calculated from comparison of the stigmasterol/cholesterol ratio at the beginning of the reaction (t=0, before adding the enzyme) and the same ratio after 14 hours. Seven different lipase preparations were used in this study. The results of this study are given in Table 9, below, and are expressed as % (w/w) conversion of free stigmasterol to stigmasterol esters.

TABLE 9
% Conversion of stigmasterol to stigmasterol oleate using
different enzymatic preparations

| | Conversion of |
|---|---|
| Enzyme preparation | stigmasterol (% w/w) |
| Blank (no enzyme added) | 0 |
| Enzymoalc 4B | 65 |
| Enzymoalc 4C | 70 |
| Enzymoalc 4D | 71 |
| Crude Enzymoalc 4 | 20 |
| Crude Enzymoalc 4 immobilized | 21 |
| on silica | |
| Crude Enzymoalc 7 | 60 |
| Enzymoalc 7B | 64 |
| Enzymoalc 7G | 84 |

It may be seen from Table 9 that the conversion of stigmasterol to its oleic acid ester using the modified-immobilized enzymes 4 and 7 (applying different matrices) is much higher with Enzymoalc4 and higher with Enzymolac7, comparing to the conversion when using crude enzyme.

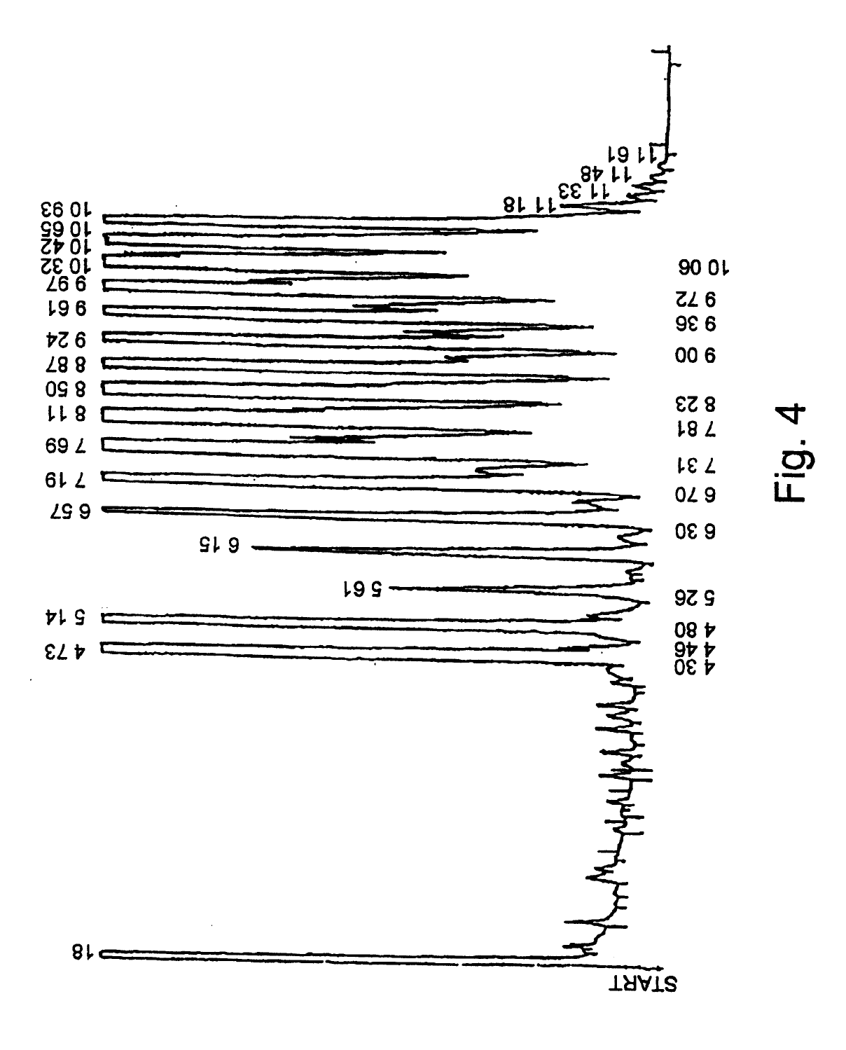

FIG. 4 shows the AMF enriched with stigmasterol (peak at 5.14) and with cholesterol (peak at 4.73) as an internal standard before adding the enzyme. It can be seen that the concentrations of both the cholesterol and stigmasterol are almost the same.

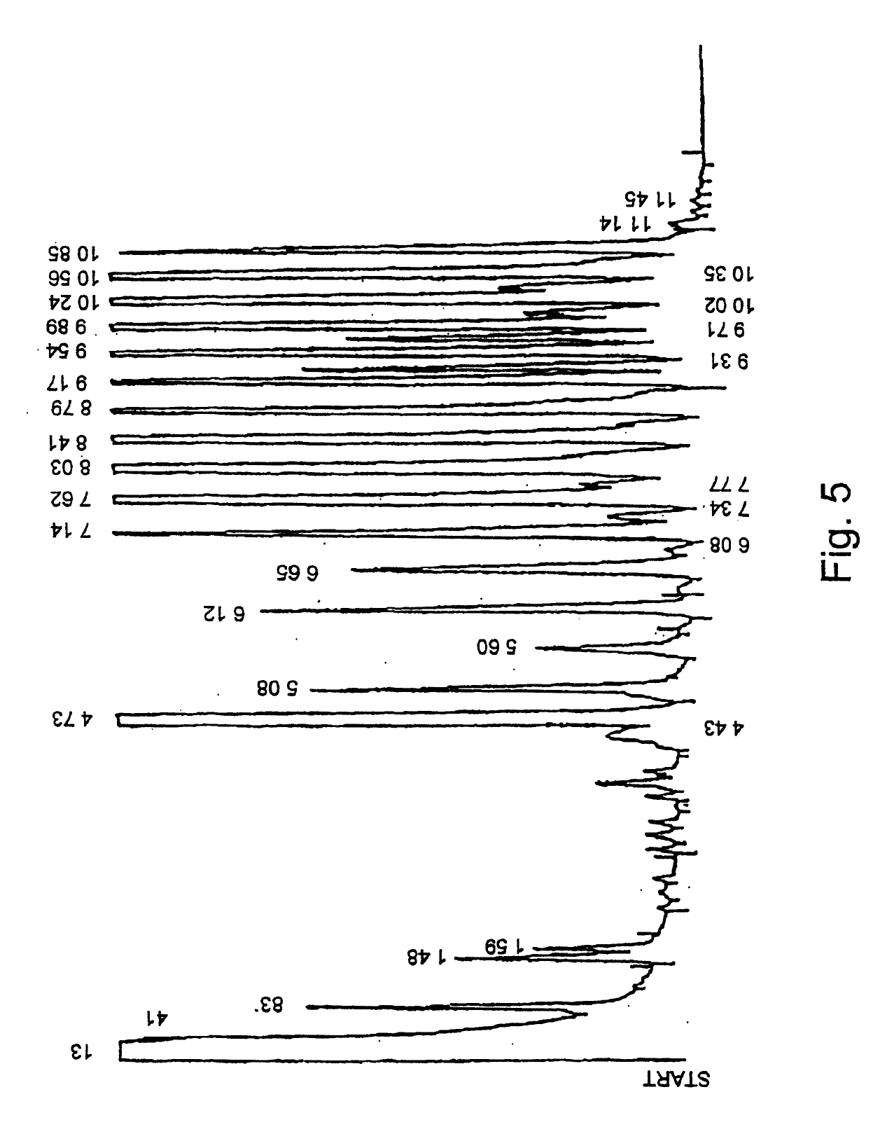

FIG. 5 shows the AMF sample after treatment with Enzymoalc 7c. The cholesterol concentration is the same as in FIG. 4 but as can be seen the concentration of stigmasterol is much lower than its concentration as appearing in FIG. 4.

### EXAMPLE 6

Lipase-Catalyzed Alcoholysis of Stigmasterol in Organic Solvents

The lipase-catalyzed alcoholysis of stigmasterol in olive oil was performed using the following reaction conditions:

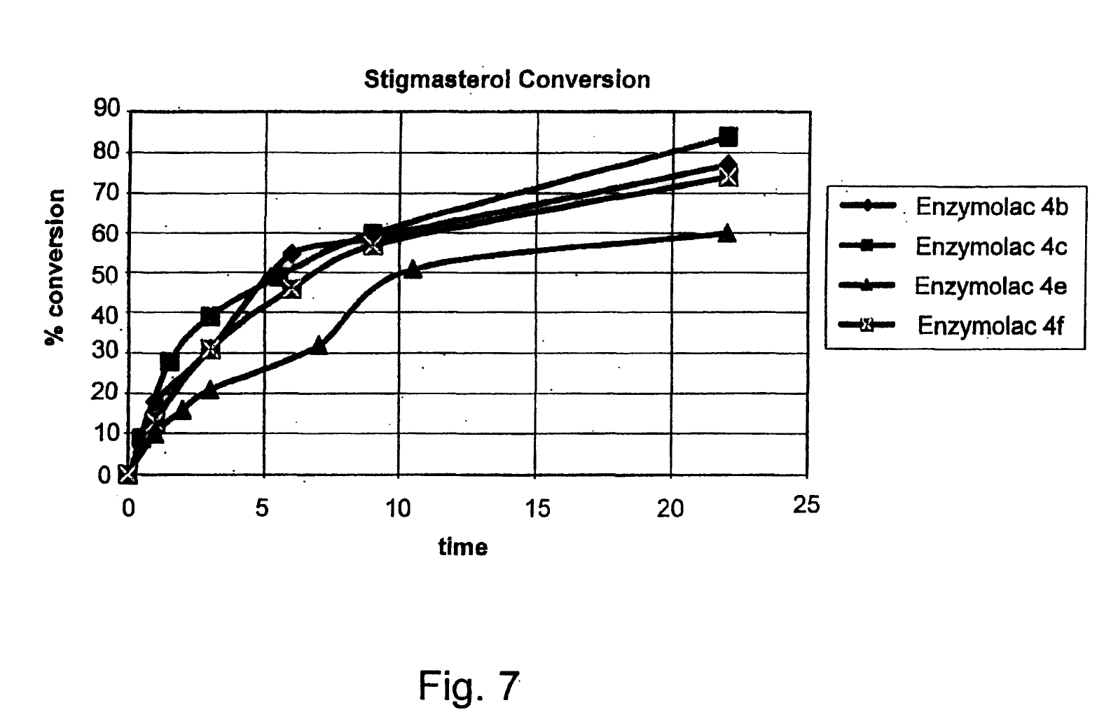

100 mg of a lipase preparation were added to 0.5 ml olive oil enriched with 50 mg stigmasterol in 1.5 ml n-hexane. The reaction mixture was then heated to 60° C and incubated for 22 hours with shaking at 60° C. Samples were taken at pre-determined time intervals, diluted with n-hexane (50 μl reaction mixture/300 μl n-hexane) and injected into a gas chromatography system, as described above. Table 10 and FIG. 7 summarize the % conversion of stigmasterol to the corresponding oleate ester with the different lipase preparations.

TABLE 10
Conversion of stigmasterol to stigmasterol oleate in
olive oil in the presence of hexane using enzymes
preparations 4B, 4C, 4E, 4F

| Time | | | | |
|---|---|---|---|---|
| (hrs) | Enzymoalc 4B | Enzymoalc 4C | Enzymoalc 4E | Enzymoalc 4F |
| 0 | 0 | 0 | 0 | 0 |
| 0.5 | 9 | 9 | | |
| 1 | 18 | | 10 | 13 |
| 1.5 | | 28 | | |
| 2 | | | 16 | |
| 3 | 31 | 39 | 21 | 31 |
| 5.5 | | 49 | | |
| 6 | 55 | | | 46 |
| 7 | | | 32 | |
| 9 | 59 | 60 | | 57 |
| 10.5 | | | 51 | |
| 22 | 77 | 84 | 60 | 74 |

### EXAMPLE 7

Lipase-Catalyzed Alcoholysis of Stigmasterol Solvent-Free System

The lipase-catalyzed conversion of stigmasterol to stigmasterol ester in Olive oil in a solvent-free system was performed using the following reaction conditions:

100 mg of a lipase preparation were added to 1 ml olive oil enriched with 30 mg stigmasterol. The reaction mixture was then heated to 60° C. for 14 hours. Following this period, samples were taken, diluted with n-hexane (40 mg reaction mixture/600 μl n-hexane) and injected into a gas chromatography system, as described above.

Tables 11 and 12 give the quantitative results for stigmasterol conversion in olive oil, with different enzyme preparations.

TABLE 11
Stigmasterol conversion to stigmasterol oleate using different
preparations of enzymoalc4

| Preparation Enzymatic | % Conversion of Stigmasterol |
|---|---|
| Crude enzymoalc4 | 12 |
| Enzymoalc 4E | 85 |
| Enzymoalc 4B | 97 |
| Eazymoalc 4F | 82 |
| Enzymoalc 4C | 95 |
| Enzymoalc 4D | 90 |
| Enzymoalc 4G | 72 |

TABLE 12
Stigmasterol conversion to stigmasterol oleate using different
preparations of enzymoalc7

| Enzymatic preparation | Stigmasterol % Conversion of |
|---|---|
| Crude Enzymoalc 7 | 17 |
| Enzymoalc 7D | 42 |
| Enzymoalc 7G | 72 |

Both Enzymoalc4 and Enzymoalc7 preparations were highly active, giving in the case of Enzymoalc4, more than 95% stigmasterol conversion under conditions in which no solvent is added.

### EXAMPLE 8

Enrichment of Olive Oil with a Mixture of Phytosterols

Olive oil was enriched with 10% (w/w) phytosterol mixture ADM product code 040095, containing 90% phytosterols (among them are: beta-sitosterol, campesterol, stigmasterol, brassicasterol and sitostanol).

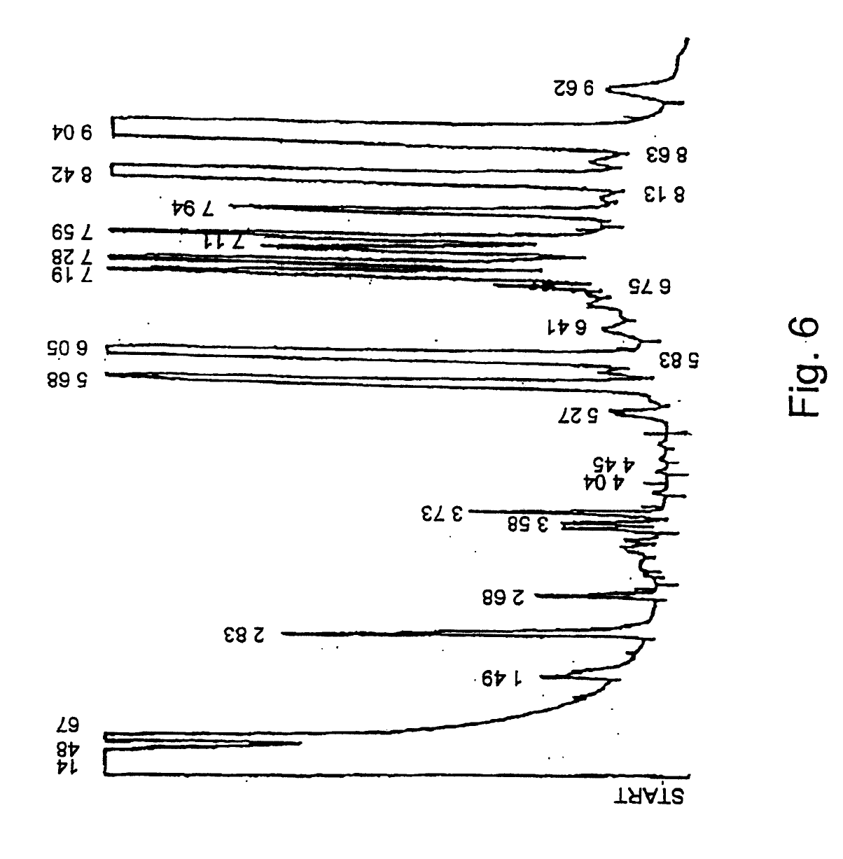

FIG. 6 shows the reaction products after an overnight reaction. The retention time of the phytosterol esters are in the region of 7.69-7.10 minute whereas the retention time of the phytosterols are in the region of 3.73-3.01 minute. It can be seen from FIG. 6 that the ester-content is much higher than the alcoholic reactant. The diglyceride content in the olive oil was slightly increased after the alcoholysis reaction.

### EXAMPLE 9

Simultaneous Neutralization of Free Butterfat Cholesterol and Enrichment of the Butterfat with Stigmasterol

The lipase-catalyzed conversion of stigmasterol to stigmasterol ester and simultaneously neutralization of the cholesterol in AMF was performed using the following reaction conditions:

100 mg of a lipase preparation were added to 1 ml ANF enriched with 20 mg stigmasterol. The reaction mixture was then heated to 40° C. for 14 hours. Following this period, samples were taken, diluted with n-hexane (40 mg reaction mixture/600 μl n-hexane) and injected into a gas chromatography system, as described above.

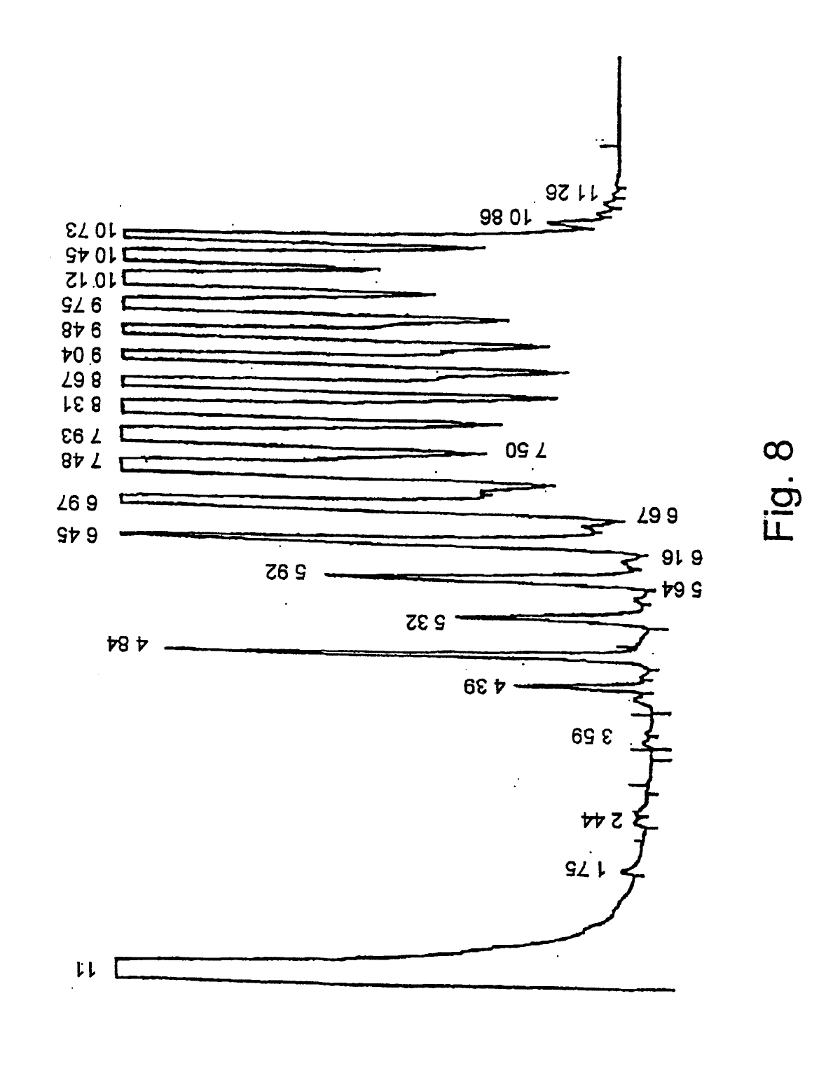

FIG. 8 shows the ANY enriched with stigmasterol (peak at 4.84), before adding the enzyme. The cholesterol peak appears at retention time of 4.39 min.

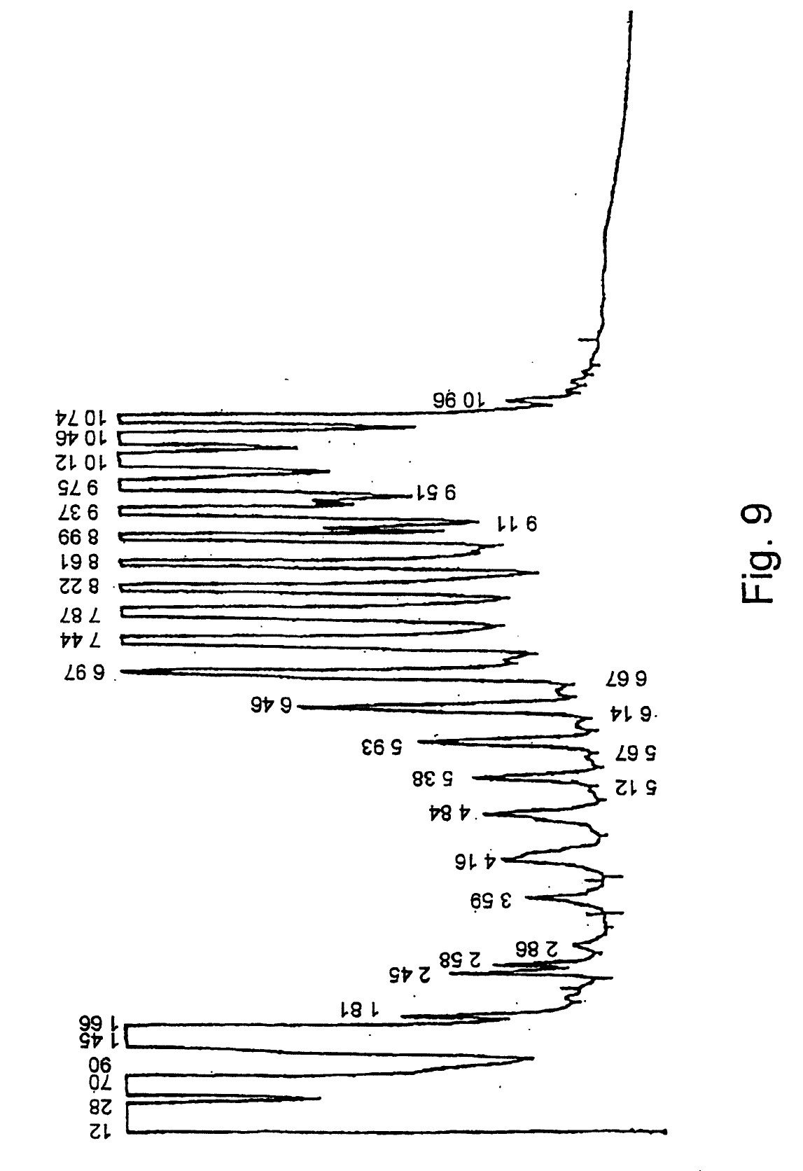

FIG. 9 shows the AMF sample after the enzymatic reaction. It can be seen that the cholesterol peak disappears and the intensity of the stigmasterol peak decreases.

This example demonstrates the enzyme ability to simultaneously neutralize the cholesterol and esterify the stigmasterol when both are present in the same sample, while the sample fat structure remains unchanged or is only slightly modified.

While specific embodiments of the invention have been described for the purpose of illustration, it will be understood that the invention may be carried out in practice by skilled persons with many modifications, variations and adaptations, without departing from its spirit or exceeding the scope of the claims.

1. Process for the selective alcoholysis of a free sterol, contained in, or externally added to a fat-based product, in an organic solvent or in a solvent-free system in the presence of an immobilized-surfactant coated lipase complex, said complex possessing a high level of sterol-specific selective alcoholytic activity and minimal acidolytic and transesterification activities, for a time sufficient for alcoholysis of said free sterol to occur, following which the immobilized-surfactant coated lipase complex is optionally removed; wherein said selective alcoholysis process, provides a fat-based product containing a reduced amount of free sterol, in particular, cholesterol.

2. Process according to claim 1, wherein the sterol is cholesterol.

3. Process according to claim 1, wherein the sterol is a phytosterol.

4. Process according to claim 3, wherein the phytosterol is stigmasterol.

5. Process according to claim 1, wherein the immobilized-surfactant coated lipase complex is in a granular form.

6. Process according to claim 1, wherein the fat-based product is a food or a nutritional product.

7. Process according to claim 1, wherein the fat-based product is a cosmetic or cosmetic-related product.

8. Process according to claim 1, wherein the cholesterol ester may optionally be removed from said lipase complex.

9. Process according to claim 2, wherein the sterol is a constituent of butterfat.

10. Process according to claim 9, wherein the alcoholysis reaction is carried out in an organic solvent.

11. Process according to claim 10, wherein the organic solvent is n-hexane.

12. Process according to any one of claims 1 to 9, wherein the reaction is performed in an organic solvent-free system.

13. Process according to any one of claims 1 to 12, wherein the lipase is selected from the group consisting of Candida antarctica, Candida antarctica A and Candida rugosa.

14. Process according to any of claims 1, 2 and 8, for preparing a substantially cholesterol-free fat-based product, comprising esterifying the cholesterol contained in a precursor of said product by said immobilized-surfactant coated lipase, and optionally removing the resulting esterified cholesterol from the lipase complex.

15. Process according to claim 14, wherein the substantially cholesterol-free product is a butterfat-containing product.

16. Process according to claims 14 and 15, wherein the lipase is immobilized onto an insoluble matrix.

17. Process according to claims 14 and 15, wherein the lipase is immobilized onto an insoluble matrix.

18. Process according to claim 14, wherein the substantially cholesterol-free product is a food preparation.

19. A modified butterfat composition wherever produced by the process according to claims 1, 2 and 8.

20. A low cholesterol food preparation containing a modified butterfat composition according to claims 14, 15 and 19.

21. A low cholesterol food preparation according to claim 20, wherein said food preparation is selected from the group consisting of low-cholesterol butter, cocoa butter, anhydrous milk fat, ice cream, coffee whiteners and creams, cheeses, other dairy products, other cholesterol-containing foods and phospholipids containing foods.

22. Process according to any one of claims 1 to 18, wherein the lipase is selected from the group consisting of Aspergillus niger, Candida antarctica, Candida cylindracea, Mucor miehei, Pseudomonas cepacia, Rhizopus niveus, Rhizopus arrhizus, Hog pancreas, Aspergillus oryzae, Candid lipolytica, Mucor javanicus, Pencillium roqueforti, Pseudomonas fluorescens, Rhizomucor miehei, wheat germ, Chromobact. viscosum, lipoprotein from Pseudomonas species, lipoprotein from Pseudomonas species B, Lilipase A-10FG, Saiken 100, Lipase G, Lipase M, Lipase F-AP-15, Newlase F, Pancreatin F, Newlase AP6, Lipase AY, Lipase PS, Novozym 525, Novozym 868, Novozym 388, Novozym 398, Novozym 435 (immobilized), Lipozyme IM-60 (immobilized) and PPL.

23. Process for the in situ enrichment of a fat-based product with esterified phytosterol ester(s), comprising the steps of:
(a) adding at least one phytosterol to a fat-based product;
(b) adding to the mixture of (a) an immobilized-surfactant coated lipase complex possessing a high level of sterol-specific, selective alcoholytic activity and minimal acidolytic and interesterfication activities;
(c) incubating the reaction mixture, for a time sufficient for maximal alcoholysis of said free sterol to occur;
(d) optionally removing the immobilized lipase complex upon completion of the reaction; and
(e) optionally removing the esterified cholesterol obtained in (d).

24. Process for the in situ enrichment of a fat-based product with esterified phytosterol ester(s) according to claim 23, wherein the fat-based product is a food or a nutritional product.

25. Process for the in situ enrichment of a fat-based product with esterified phytosterol ester(s) according to claim 23, wherein the fat-based product is a cosmetic or cosmetic-related product.

26. Process according to claim 23, wherein the esterification of said phytosterol is simultaneously accompanied by esterification of any free cholesterol present in said fat-based product.

27. Process according to claim 23, wherein the esterification of said phytosterol is performed before, or following the esterification of cholesterol present in said fat-based product.

28. Process according to any one of claims 23 to 25, wherein the phytosterol is stigmasterol.

29. A fat-based product enriched with esterified phytosterol ester(s), prepared by the process according to claim 23.

30. A fat-based product enriched with esterified phytosterol ester(s), and/or depleted in cholesterol level, prepared by the process according to claim 23.

31. A food preparation enriched with esterified phytosterol ester(s), prepared by the process according to claim 30, wherein said food preparation is selected from the group consisting of low-cholesterol butter, cocoa butter, anhydrous milk fat, ice cream, coffee whiteners and creams, cheeses, other dairy products, other cholesterol-containing foods, and phospholipids-containing foods.

32. Cosmetics and/or cosmetics-related products enriched with esterified phytosterol ester(s), obtained by the process according to claim 25.

33. (New) Process for the selective esterification of a free sterol, contained in, or externally added to a fat-based product, with the addition of carboxylic fatty acid(s) and/or ester derivative(s) thereof in an organic solvent or in a solvent-free system in the presence of an immobilized-solvent or in a solvent-free system in the presence of an immobilized-surfactant coated lipase complex, said complex possessing a high level of sterol-specific, selective esterification activity and minimal acidolytic and transesterification activities for a time sufficient for esterification of said free sterol to occur, following which the immobilized-surfactant coated lipase complex is optionally removed; wherein said selective esterification process, provides a fat-based product containing a reduced amount of free sterol, in particular, cholesterol.

34. (New) Process for the in situ enrichment of a fat-based product with esterified phytosterol esters(s), comprising the steps of:
(a) adding at least one phytosterol to a fat-based product;
(b) adding to the mixture of (a) at least one carboxylic fatty acid or ester derivative thereof;
(c) adding to the mixture of (b) an immobilized-surfactant coated lipase complex possessing a high level of sterol-specific esterification activity and minimal acidolytic and interesterification activities, said complex optionally being surfactant-coated;
(d) incubating the reaction mixture, for a time sufficient for maximal esterification of said free sterol to occur;
(e) optionally removing the immobilized lipase complex upon completion of the reaction; and
(f) optionally removing the esterified cholesterol obtained in (d).
